(19) United States
(12) Patent Application Publication
(10) Pub. No.: US 2011/0105617 A1
(43) Pub. Date: May 5, 2011

(54) METHOD FOR TREATMENT OF NEOPLASTIC DISEASES WITH ALPHA-KETO GLUTARATE AND/OR SALTS THEREOF

(75) Inventors: Stefan G. Pierzynowski, Malmo (SE); Martyna Kandefer-Szerszen, Lublin (PL); Wojciech Rzeski, Lublin (PL)

(73) Assignee: SGP & SONS AB, Lund (SE)

(21) Appl. No.: 12/940,573

(22) Filed: Nov. 5, 2010

Related U.S. Application Data
(63) Continuation-in-part of application No. 12/161,538, filed on Jul. 18, 2008.
Application No. 12/161,538 is a continuation-in-part of application No. 11/813,526, filed on Oct. 23, 2007, now Pat. No. 7,759,396.

(30) Foreign Application Priority Data
Jan. 11, 2005 (PL) 372183
Jan. 19, 2006 (SE) 0600130-9

(51) Int. Cl. A61K 31/194; A61P 35/00

(52) U.S. Cl. 514/574

(57) ABSTRACT

A method for treatment or prophylaxis of neoplastic diseases includes administering an antineoplastic preparation to a subject in need thereof. The active component of the antineoplastic preparation is alpha-ketoglutarate and/or a salt of alpha-ketoglutarate.

### CROSS-REFERENCE TO RELATED APPLICATIONS

This application is a continuation-in-part of U.S. Application No. 12/161,538, filed Jul. 18, 2008, which is the U.S. national phase under § 371 of International Application No. PCT/EP2007/050499, filed Jan. 18, 2007 and a continuation-in-part of U.S. Application No. 11/813,526, filed Oct. 23, 2007 (now U.S. Pat. No. 7,759,396), which is the U.S. national phase under § 371 of International Application No. PCT/PL2006/000003, filed Jan. 10, 2006. The entire disclosures of all the foregoing applications are hereby incorporated by reference.

### BACKGROUND OF THE INVENTION

1. Field of the Invention

The present invention relates to methods for treatment of neoplastic diseases. More particularly, the present invention relates to use of alpha-ketoglutarate and/or salts thereof in such treatments.

2. Description of the Related Art

Neoplastic diseases are one of the most significant health problems throughout the world, and the increased number of cancer cases reported around the world is a major concern.

Helicobacter pylori is estimated to be responsible for up to 90% of the cases of peptic ulcer disease (PUD) afflicts over 10% of the US population sometime in their lifetime. Estimates for worldwide prevalence of H. pylori infections range from 300 million to over two-thirds of the world's population. H. pylori infection is also associated with 650,000 annual cancer deaths worldwide from gastric adenocarcinoma.

The following documents were cited in the prosecution of U.S. Application No. 11/813,526 and appear as References Cited on the cover of U.S. Pat. No. 7,759,396:

U.S. Pat. No. 5,006,551

U.S. Pat. No. 6,429,229

U.S. Patent Publication No. 2004/0259766

International Publication No. WO 2004/012662

International Publication No. WO 2004/098619

International Publication No. WO 2006/066244

Robinson et al., Amino acid nutrition and immune function in tumour-bearing rats . . . Clinical Science, 1999, vol. 97, pp 657-669.

### SUMMARY OF THE INVENTION

An object of the invention is antineoplastic preparation and the use of antineoplastic preparation.

In spite of the significant progress in the chemotherapy, radiotherapy and immunotherapy in last years the problem of the effective antineoplastic therapy is still the serious challenge in a present medicine. Epidermiological research indicates that in countries fully developed one of three people suffers from the different neoplastic diseases. In that group each fourth case is a lethal one. Cytostatics actually used in treatment can have side effects thereby limiting the effectiveness and decreasing the quality of a patient's life.

Therefore the development of new drugs acting specifically on the neoplastic cells with the simultaneous protective activity on the normal cells is an especially urgent challenge.

The antineoplastic preparation distinguishes itself in that it contains alpha-ketoglutarate (AKG) or/and glutamine or/and glutamate or/and alpha-ketoglutarate of ornithine or/and dipeptides of glutamine and other amino acids or/and tripeptides of glutamine and other amino acids or/and di- and tripeptides of glutamate with other amino acid or/and mono- and divalent metal salts and other of alpha-keto-glutarate or/and glutamine or/and glutamate or/and ornithine of alpha-ketoglutarate.

The use of antineoplastic preparation is that the preparation containing alpha-ketoglutarate (AKG) or/and glutamine or/and glutamate or/and alpha-ketoglutarate of ornithine or and dipeptides of glutamine and other amino acids or/and tripeptides of glutamine and other amino acids or/and di- and tripeptides of glutamate with other amino acid or/and mono- and divalent metal salts and other of alpha-keto-glutarate or/and glutamine or/and glutamate or/and ornithine or alpha-ketoglutarate is used in the prophylaxis of neoplastic diseases

Other use of antineoplastic preparation is that the preparation containing alpha-ketoglutarate (AKG) or/and glutamine or/and glutamate or/and alpha-ketoglutarate of ornithine or/and dipeptides of glutamine and other amino acids or/and tripeptides of glutamine and other amino acids or/and di- and tripeptides of glutamate with other amino acid or/and mono- and divalent metal salts and other of alpha-keto-glutarate or/and glutamine or/and glutamate or/and ornithine of alpha-keto-glutarate is used for metastases inhibition.

The preparation and the use of the preparation allows for the inhibition of the migration of neoplastic cells reflecting the potential role of AKG in the metastases inhibition. The preparation added to the diet plays a role as neo-adjuvant supporting existing methods used in the neoplasm treatment. It may improve the quality of the a patient's life through the synergistic action with antineoplastic drugs and simultaneous protective activity to normal cells.

According to an embodiment of the invention, there is provided a use of a substance comprising at least one member is selected from the group consisting of alpha-ketoglutaric acid (AKG) and derivates, metabolites, analogues or salts thereof for the manufacture of a pharmaceutical preparation or a food or feed supplement for the treatment or prophylaxis of diseases and conditions associated with low levels of AKG in a human or animal with low levels of AKG compared with normal average AKG levels.

According to a further embodiment of the invention, said diseases and conditions associated with low levels of AKG include neoplastic diseases and diseases or conditions related to infection of pathogenic strains of Helicobacter pylori, particularly diseases of the gastrointestinal tract including diseases such as gastric cancer, and gastric mucosa-associated lymphoid tissue lymphoma.

According to a further embodiment of the invention the amount of AKG given to a human or animal is a therapeutically effective amount.

In specific embodiments the therapeutically effective amount is in the interval from 1 to 1000, 10 to 400, or 10 to 100 mg/kg body weight/day.

According to a further aspect of the present invention, there is provided a for the treatment or prophylaxis of diseases and conditions associated with low levels of AKG in a human or animal with low levels of AKG compared with normal average AKG levels, which method comprises administering to a subject in need for such treatment or prophylaxis of an effective amount of at least one member selected from the group consisting of alpha-ketoglutaric acid (AKG) and derivates, metabolites, analogues or salts thereof. According to one embodiment of the invention, alkali or alkaline earth metal salts of alpha-ketoglutaric acid are used.

According to an embodiment of the invention, said diseases and conditions associated with low levels of AKG include neoplastic diseases and diseases or conditions related to infection of pathogenic strains of Helicobacter pylori, particularly diseases of the gastrointestinal tract including diseases such as gastric cancer, and gastric mucosa-associated lymphoid tissue lymphoma.

### BRIEF DESCRIPTION OF THE DRAWINGS

FIG. 1 depicts a curve presenting the proliferation of A549 cells with the stimulation by AKG.

FIG. 2 depicts the proliferation of C6 cells with AKG.

FIG. 3 depicts the proliferation of HT-29 cells with AKG stimulation

FIG. 4 presents the proliferation of human neoplastic cells A549 in the presence of the cyclophosphamide with AKG.

FIG. 5 depicts the proliferation of human neoplastic cells A549 in the presence of iphosphamide with AKG.

FIG. 6 depicts the proliferation of human neoplastic cells A549 in the presence of thiotepa with AKG

FIG. 7 depicts the inhibition of C6 cells migration due to AKG.

### DETAILED DESCRIPTION OF THE PREFERRED EMBODIMENT

### The Cultures of Neoplastic Cells

A549—human neoplastic cells of the lung cancer; the continuous line obtained from the Institute of the Immunology and the Experimental Therapy of Polish Academy of Science in Wroclaw.

HT-29—human neoplastic cells of the large intestine cancer; the continuous line obtained from the Institute of the Immunology and the Experimental Therapy of Polish Academy of Science in Wroclaw.

C6—the rat neoplastic cells of the brain cancer (glioma); the continuous line obtained from the Department of Neonatology, Humboldt University, Berlin, Germany.

### The Bases of the Culture

Cells of the A549 line were cultured on the basis DMEM:F-12 HAM (2:1), HT-29 and C6 cells on the basis DMEM. To the culture basis, 10% foetal beast serum (FBS), penicillin 100 i.u./ml and streptomycin 100 .mu.g/ml were added. The basis DMEM:F-12 HAM, DMEM was produced by Sigma company (Sigma, St. Louis, Mo., U.S.A.). The foetal beast serum (FBS) was produced by Life Technologies company (Life Technologies, Karlsruhe, Germany). Remaining reagents were produced by Sigma company.

### The Preparation of Cellular Cultures

Cells stored in liquid nitrogen in a tissue bank were unfrozen at a temperature of 37.degree. C., then poured into the plastic bottles containing the proper basis. They were cultured in a temperature of 37.degree. C. in the incubator with 5% CO.sub.2 flow. After the cells' reproduction liquid was poured out, the cells were washed with PBS (without the calcium and magnesium ions) and processed with 0.25% trypsin solution+EDTA to receive the suspension of cells necessary in the experience.

### Assessment of the Antiproliferative Activity of AKG in the Cellular Culture

Prepared earlier, in the culture basis, a suspension of cells with a density 1.times.10.sup.4 cells/ml (A549), 4.times. 10.sup.4 cells/ml (HT-29) and 0.5.times.10.sup.4 cells/ml (C6) was poured into the 96-pits microplate with the flat bottom (NUNC company, Roskilde, Denmark) in the volume 100 .mu.1/pit. After sticking of cells (24 hours) the liquid was carefully pulled down and then different concentrations of AKG and examined cytostatics (cyclophosphamide, iphosphamid, thiotepa) in liquid with 10% FBS (100 .parallel.1/pit) were added. The cultures on plates were left for 96 hours incubation at a temperature of 37.degree. C., in the atmosphere 95% air and 5% CO.sub.2. The antiproliferative activity of examined substances was assessed with the method MTT.

### The Method MTT (According to Kit “Cell Proliferation Kit III”, Boehringer Manheim)

This method was worked out to determine the proliferation and vitality of cells in studies on the cytotoxic and antiproliferative substances. In metabolically active cells tetrazolic yellow salt MTT is reduced to formazane blue with the mitochondrial dehydrases. Formazane crystals, insoluble in water, accumulate in cells and for their dissolutions the use of organic detergent, breaking the membrane and simultaneously solvent the dye, is necessary. For this purpose the buffer SDS-HC1 with pH 7.4 is used. The concentration of released dye is evaluated quantitatively in the reader for 96-pits plates at the wavelength 570 nm. The colour intensity is directly proportional to the quantity of alive cells.

MTT solution in PBS condition in concentration 5 mg/ml was added into each pit on a plastic plate in a dose of 15 .mu.1/pit. Plates were incubated for 3 hours at a temperature of 37.degree. C. Thereafter, buffer SDS-HC1 in a does 100 .mu.1/pit was added and the plates were left at a temperature of 37.degree. C. all night. The results were evaluated next day with the use of E-max Reader (Molecular Devices Corporation, Menlo Park, Calif., U.S.A.).

Evaluation of Cells Migration Degree with the Method “Wound Assay”

This method is for the estimation of the activity of substances affecting the mobility of cells in vitro. It is used in research on wounds healing, angiogenesis and neoplastic metastases.

C6 cells (1.times.10.sup.6) suspended in the culture basis with the addition of 10% serum (FBS) were poured onto the culture plates (NUNC, Roskilde, Denmark) with 4 cm diameter. Next day in the equal layer of cells the flaw (wound) was done with the ending of automatic pipette and unstuck cells were removed by twice-rinsing the plates with a PBS solution. Then, AKG (10 and 20 mM) dissolved in the culture basis was added to the prepared culture. Plates were incubated for 24 hours in a temperature of 37.degree. C. and in a humid atmosphere 95% air and 5% CO.sub.2. Thereafter the cultures were coloured with the May-Grunwald-Giemza method. Then the microscopic analysis was performed with the microscope Olympus BX51 (Olympus Optical CO., LTD, Tokyo, Japan) with the use of the software analySIS.RTM. (Soft Imaging System GmbH, Munster, Germany). The degree of cells migration was assessed in cytometry as the number of cells which populated the wound done earlier in the layer of cells. At least 50 chosen fields on 8 photographs were assessed.

### Results: The Estimation of Antiproliferative Activity of AKG

Antiproliferative activity of AKG in the cultures was estimated in differen kinds of neoplastic cells: the lung cancer cells (A549), the large intestine cancer cells (HT-29) and glioma cells (C6). Cells were processed with AKG in concentrations 0.5, 1, 2.5, 5, 10 and 20 mM, for 96 hours.

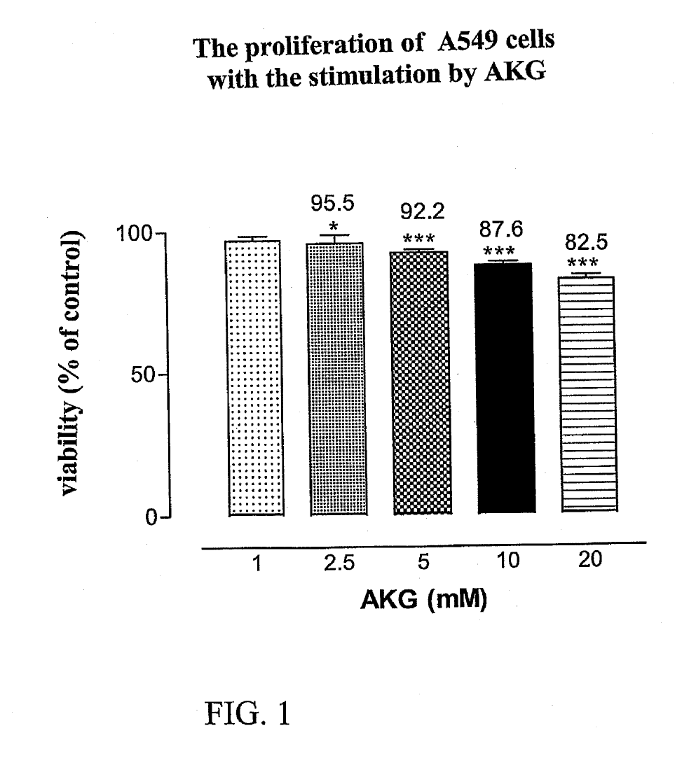

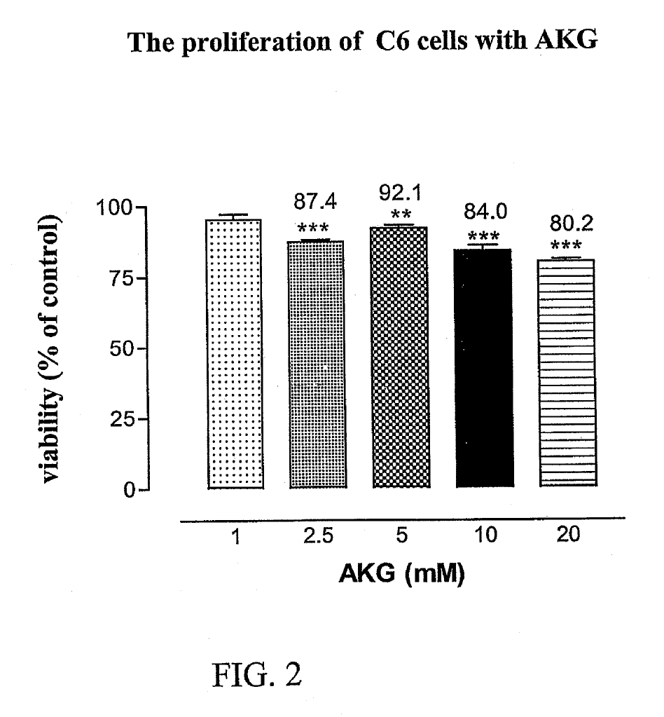

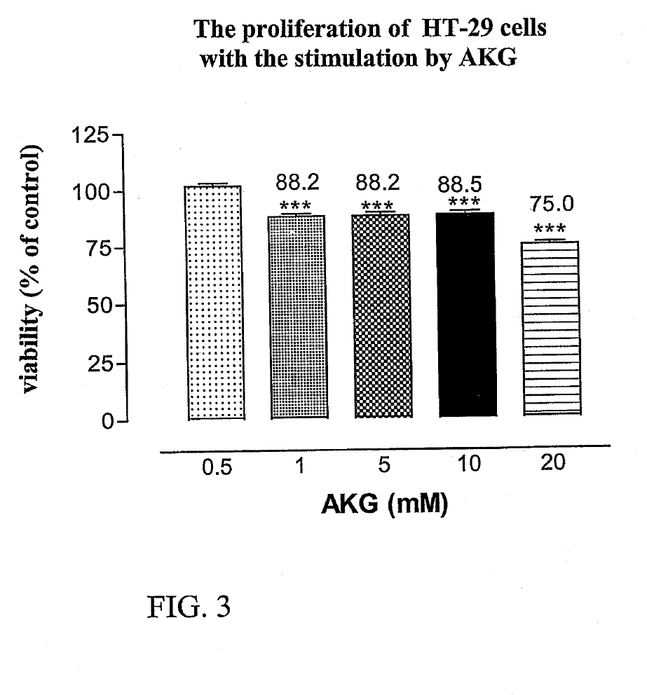

The examined substance has had antiproliferative activity with relation to all neoplastic cells types (see the diagram—FIG. 1, FIG. 2, FIG. 3). Statistically significant (4.5%) inhibition of cells' growth was observed at AKG concentration 2.5 mM in A549 cell line in the comparison to the control group. That effect was correlated with a dose of AKG and was 7.8%, 12.4%, 17.5% with doses 5 mM, 10 mM and 20 mM respectively (see the diagram on FIG. 1). The growth inhibition in glioma cells (C6) was 12.6% with AKG dose 2.5 mM, 7.9%—5 mM, 16%—10 mM and 19.8%—20 mM, respectively. The growth inhibition of large intestine cancer cells (HT-29) hadn't had the lineal character with AKG concentrations between 1 and 10 mM. The dose 1 mM inhibited the cells growth by 11.8%. The similar effect was obtained with a dose 5 mM (11.8%) and 10 mM (11.5%). Only a dose 20 mM caused the significant (25%) inhibition of these cells' growth (the diagram—FIG. 3).

### The Estimation of the Interaction Between AKG and Antineoplastic Drugs

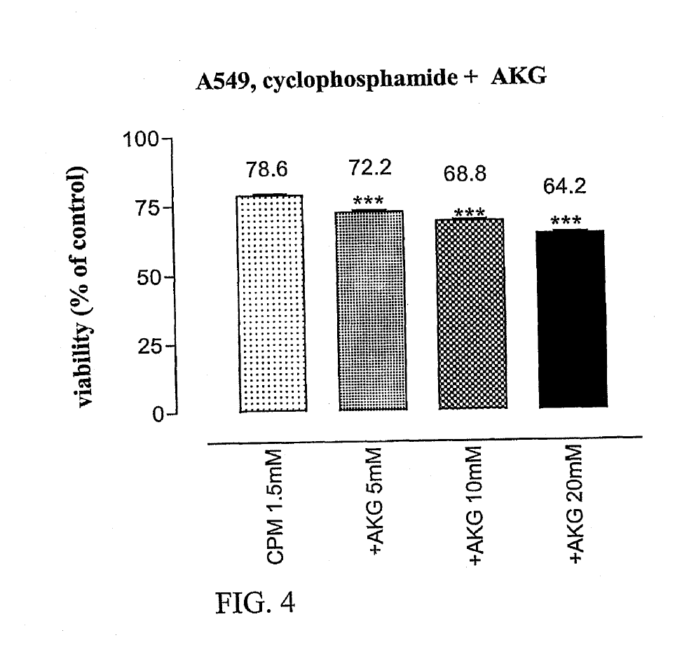

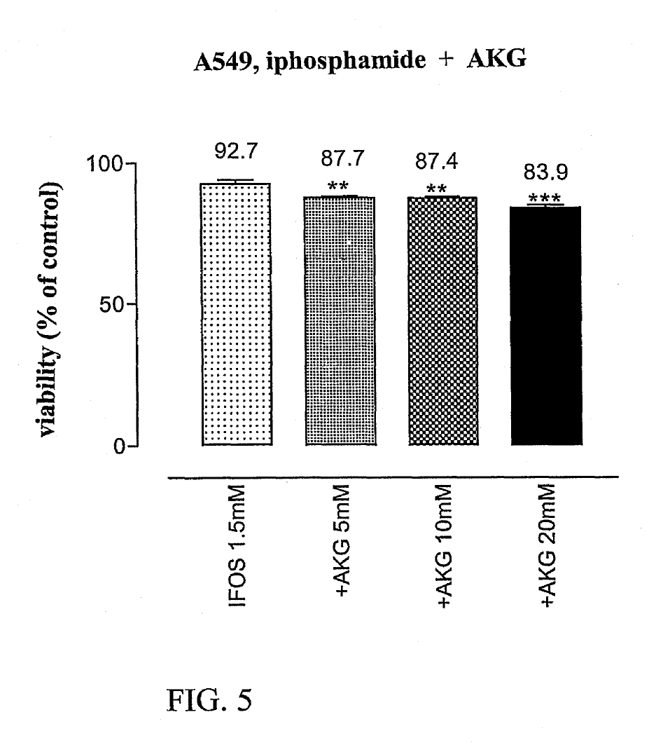

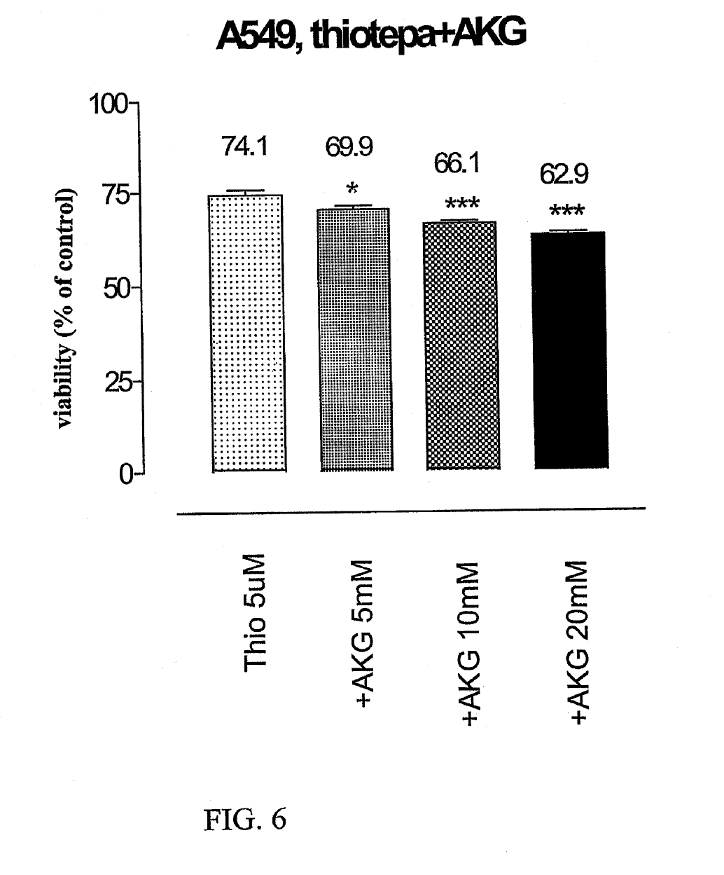

The research on the interaction between AKG and popular cytostatics used in cancer chemotherapy was performed in the culture of lung cancer cells (A549). For that purpose cells were treated with the following cytostatics: cyclophosphamide (1.5 mM), iphosphamid (1.5 mM) and thiotepa (5 .mu.M), alone and in combination with AKG (5, 10 and 20 mM). The additive effect of AKG on cytostatic activity of used chemotherapeutics was observed (results are presented on FIG. 4, FIG. 5 and FIG. 6). Cyclophosphamide in a concentration 1.5 mM inhibited the growth of A549 cells by 21.4%. The addition of AKG increased its cytostatic activity by 6.4%, 9.8% and 14.4% respectively (the diagram—FIG. 4). Iphosphamid (1.5 mM) inhibited the cells growth by 7.3%. After AKG addition (5, 10 and 20 mM) that effect increased by 5%, 5.3% and 8.8% respectively (the diagram—FIG. 5). AKG intensified also cytostatic activity of thiotepa (5 .mu.M—25.9%) by 4.2% (5 mM), 8% (10 mM) and 11.2% (20 mM) (results are presented on FIG. 6).

### The Influence of AKG on the Migration of Neoplastic Cells

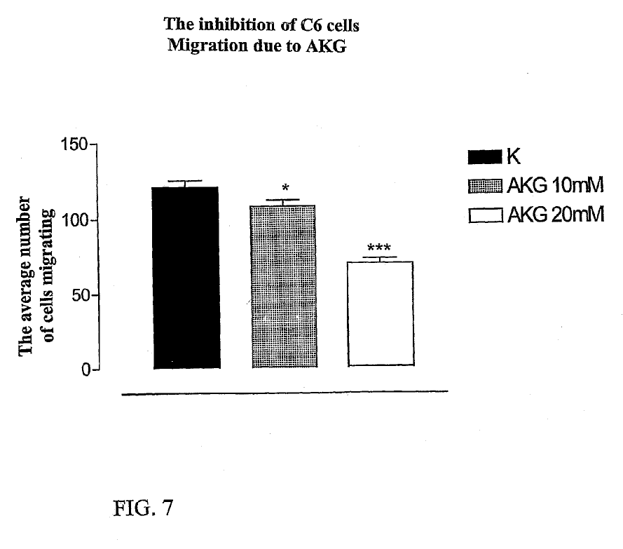

The research on the mobility of neoplastic cells was passed in the “wound assay” model. At the photograph 1 the wound in the layer of C6 cells (A), the wound population with cells after 24 hours of incubation without AKG (B) and significant inhibition of cells migration in the presence of AKG 20 mM (C) are presented. On the diagram—FIG. 7, the average number of cells migrating to one field of flaw done in the uniform layer of cells is presented. Statistically significant inhibition of cells migration in the presence of 10 mM and 20 mM AKG was shown.

### The Statistical Analysis

The statistical analysis was performed with the use of t-student test. * p<0.05, ** p<0.01, *** p<0.001.

What is claimed is:

1. A method for treatment or prophylaxis of neoplastic diseases, comprising administering an antineoplastic preparation wherein the neoplastically-active component consists of alpha-ketoglutarate and/or salts of alpha-ketoglutarate to a subject in need thereof

2. The method according to claim 1, wherein the neoplastic disease is cancer.

3. The method according to claim 1, wherein the neoplastic disease is associated with Helicobacter pylori.

4. The method according to claim 3, wherein the neoplastic disease is gastric cancer.

5. The method according to claim 3, wherein the neoplastic disease is gastric mucosa-associated lymphoid tissue lymphoma.

6. The method according to claim 1, wherein the antineoplastic preparation consists of an alkali or alkaline earth metal salt of alpha-ketoglutarate.

7. The method according to claim 2, wherein the cancer is cancer metastases.

8. The method according to claim 1, wherein the salts are mono or divalent metal salts of alpha-ketoglutarate.

9. The method according to claim 1, wherein the cancer is cancer of the lung.

10. The method according to claim 1, wherein the cancer is glioma.

11. The method according to claim 1, wherein the cancer is cancer of the large intestine.
